(19) United States
(12) Patent Application Publication
(10) Pub. No.: US 2009/0270269 A1
(43) Pub. Date: Oct. 29, 2009

(54) NANO-SCALE FLUORO-BIOSENSORS EXHIBITING A LOW FALSE ALARM RATE FOR RAPID DETECTION OF BIOLOGICAL CONTAMINANTS

(76) Inventors: Ashok Kumar, Champaign, IL (US); Larry D. Stephenson, Champaign, IL (US); Jeremy A. Hale, Urbana, IL (US); Elizabeth J. Norton, Delft (NL)

Correspondence Address: U.S. Army Corps of Engineers;Humphreys Engineer Center, ATTN: CEHEC-OC (Kingman Building), 7701 Telegraph Road, Alexandria, VA, 22315-3860, US

(21) Appl. No.: 12/110,386

(22) Filed: Apr. 28, 2008

(51) Int. Cl. C12M 1/34; G01N 33/50; C40B 30/04

(52) U.S. Cl. 506/9; 435/287.2; 435/7.2

(57) ABSTRACT

A sensing system incorporating a nano-scale fluoro-biosensor for detection of specifically targeted bio-contaminants (targets). Select embodiments use fluorescent nanoparticles such as quantum dots (QD) conjugated to antibody fragments to form a sensor for a specific bio-contaminant based on fluorescent resonance energy transfer (FRET). A quenching dye may be used to label an analog, while a specific antibody is covalently bonded to a hydrophilic QD. Coupling of QD labeled antibodies and quencher labeled analogs provides enough proximity to produce appreciable FRET-based quenching. Any addition of the target displaces the dye-labeled bacteria, eliminating FRET-based quenching and results in a concentration-dependent increase in QD photoluminescence. Applications include rapid detection and identification, with a high degree of specificity and sensitivity, of a broad range of targets, including viral contaminants, e.g., in less than about three minutes. By using QDs of varying wavelengths the system may be adapted into a multiplexing immuno-assay.

### STATEMENT OF GOVERNMENT INTEREST

Under paragraph 1(a) of Executive Order 10096, the conditions under which this invention was made entitle the Government of the United States, as represented by the Secretary of the Army, to an undivided interest therein on any patent granted thereon by the United States. This and related patents are available for licensing to qualified licensees. Please contact Bea Shahin at 217 373-7234.

### BACKGROUND

Detection of biological contaminants is important in many environments, thus a number of sensors have been developed. These conventional sensors may be handheld or mounted in areas being monitored. The size of conventional sensors as well as their separate mechanical and electrical components increase cost as compared to integrated circuits. Sensors using nanotechnology reduce costs and provide new capabilities, such as for confined environments or those difficult to access.

Bio-molecules labeled with fluorescent dyes are used in a wide variety of scientific and technological applications. A number of examples can be found in the fields of molecular biology, cell biology, biochemistry, and medical diagnostics. These applications are based on the inherent advantages of fluorescent assays, some of which are high sensitivity, the facile introduction of fluorescent labels on a wide variety of bio-molecules (such as nucleotides, proteins, and peptides) with minimal effects in their activity, and usefulness in microscopy applications. Conventional fiber-optic evanescent fluorescence sensors detect concentrations of molecules known to absorb light of a given wavelength, λ₁, and subsequently fluoresce by emitting light having a second wavelength, λ₂. These sensors incorporate an optical fiber that is normally inserted into a liquid containing targeted molecules. The fiber is illuminated with light of wavelength λ₁. The targeted molecules within an evanescent field surrounding the fiber absorb the light of wavelength λ₁ and fluoresce to return light having wavelength λ₂. A detector coupled to the optical fiber measures the intensity of the returned light indicating the presence or quantity of targeted molecules within the evanescent field. Jenne, A. et al., Real-time Characterization of Ribozymes by Fluorescence Resonance Energy Transfer (FRET). Angewandte Chemie, International Edition, Verlag Chemie, vol. 38, No. 9, 1300-1303, May 3, 1999.

The characteristics of fluorescent dyes make them valuable tools in several fields. The potential for assays utilizing fluorescent dyes is further expanded through the use of fluorescent resonance energy transfer (FRET) reactions that can take place between fluorescent dyes and donor quencher molecules under controlled circumstances. By understanding and manipulating FRET reactions, sensitive and highly specific sensors may be developed to detect and identify a broad spectrum of biological contaminants. Pertinent references applicable to the basic technology include: Goldman, E., et al., J. Am. Chem. Soc. 2004 127, 5744-6751; Yang, L. & Li, Y.; Royal Soc. of Chem. 2005 131, 394-401; Clapp, A. et al., J. Am. Chem. Soc. 2003 126, 301-310; Bruno, J. et al.; Biochemical and Biophysical Research Communications 2002 287, 875-880 and Lakowicz, J. R.; Principles of Fluorescence Spectroscopy, 2^{nd} ed.; Kluwer Academic, New York, 1999.

Although highly desirable in terms of speed and ease of execution, there are few examples of FRET-based immunoassays. Most often studied are FRET-based PGR assays involving a fluoroscein quenched in the presence of carboxytetramethylrhodamine (TAMRA). However, immuno-assays utilizing FRET have great potential for the development of near real-time fluoro-biosensors.

Conventional evanescent fluorescence sensors are inadequate for universal use. In particular, their size limits them to sensing targeted molecules with particular fluorescent properties. Further, they are limited to detecting targeted molecules in a liquid since contaminants in a gas at room temperature spend a short period within the evanescent field, i.e., within a distance of about λ₁/4 of the optical fiber, and leave the evanescent field of the fiber before fluorescing. For example, conventional Polymerase Chain Reaction (PCR)-based sensor systems for detection of biological contaminants require about 30 minutes to detect the presence of a biological contaminant. Time delays of this magnitude may result in preventable exposure to biological contaminants. Further, these conventional sensors are expensive to acquire and maintain.

However, bioaffinity sensors labeled with fluorophores have been used to detect DNA hybridization and single-nucleotide polymorphisms. Didenko, V. V. BioTechniques, 31:1106-18, 2001.

Molecular beacons are single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore is covalently linked to the end of one arm and a quencher is covalently linked to the end of the other arm. Molecular beacons do not fluoresce when they are free in solution. However, when they hybridize to a nucleic acid strand containing a target sequence they undergo a conformational change that enables them to fluoresce brightly.

In the absence of targets, the probe is dark, because the stem places the fluorophore so close to the non-fluorescent quencher that they transiently share electrons, eliminating the ability of the fluorophore to fluoresce. When the probe encounters a target molecule, it forms a probe-target hybrid that is longer and more stable than the stem hybrid. The rigidity and length of the probe-target hybrid precludes the simultaneous existence of the stem hybrid. Consequently, the molecular beacon undergoes a spontaneous conformational reorganization that forces the stem hybrid to dissociate and the fluorophore and the quencher to move away from each other, restoring fluorescence.

Specifically, a molecular beacon, a DNA hairpin structure, is labeled with both a fluorophore and quencher. In the absence of the target DNA, the hairpin structure is closed and due to the close proximity of the fluorophore and quencher, fluorescence is quenched. In the presence of a complementary DNA strand, the hairpin secondary structure is destroyed and the fluorescence is released without quenching, i.e., the donor fluorophore transfers energy to the acceptor fluorophore, resulting in fluorescence. Multiple DNA strands may be detected at the same time by placing a quencher on one end of the molecular beacon DNA strand and two fluorophores (a donor fluorophore and an acceptor fluorophore) on die other end. Tyagi S and Kramer, F. R., Nat. Biotechnol 14, 303-308, 1996. A molecular beacon may be designed to target different DNA sequences by constructing complementary DNA strand hairpins, each with a different acceptor fluorophore, while keeping the donor fluorophore the same.

Biosensors, devices capable of detecting target ions using biological reactions, in contrast to bioaffinity sensors, can be modified to utilize fluorescence for detecting, identifying or quantifying target ions. The target ions then act as catalysts of the biosensor, making it a fluoro-biosensor. These modified biosensors, called fluorosensors in the literature, are highly sensitive. For example, U.S. Pat. No. 7,271,379 B2, Dielectric Microcavity Fluorosensors Excited with a Broadband Light Source, to Fan et al., issued Sep. 18, 2007, uses dielectric microspheres and equatorial whispering gallery modes (EWGM) in sensing applications, unlike the true fluorescent nanoparticles of the present invention. The sensor surface of the '379 patent is immobilized with a layer of molecules, such as antibodies, for the subsequent capture of analytes, such as antigens. In a direct assay configuration, antigens are conjugated with a fluorescent dye molecule. When the antigen binds with the antibody on the sensor surface, the fluorescent molecule is held sufficiently close to the microsphere surface that it is excited by evanescent light circulating in the microsphere. In a sandwich-type configuration, the antigen is first bound to the antibody on the sensor surface, and then a second layer of antibodies, labeled with a fluorescent dye, is added to bind to the captured antigens. The fluorescent molecules bound to the second layer of antibodies are excited by the evanescent field arising from light propagating in the whispering gallery modes (WGMs) of the microsphere. The resulting fluorescence from the excited dyes is collected and used as an indicator of the antigen binding events.

To distinguish from other biosensors and other fluorosensors, such as chemosensors, hereafter, biosensors of select embodiments of the present invention are termed fluoro-biosensors. For example, many fluorescent chemosensors, including fluorophore-labeled organic chelators and peptides have been developed for metal ion detection. Pearce et al., Bio-Org Med Chem Lett. 8, 1963-1968, 1998; Rurack, K., et al., A Selective and Sensitive Fluoroionophore for Hgll, Agl, and Cull with Virtually Decoupled Fluorophore and Receptor Units, J. Am. Chem. Soc. 122, 968-969, 2000; Hennrich. G., et al., U. J. Am. Chem. Soc. 121, 5073-5074, 1999; Winkler, J. D., et al., Photodynamic Fluorescent Metal Ion Sensors with Parts per Billion Sensitivity, J. Am. Chem. Soc. 120, 3237-3242, 1998: Oehme, I. & Wolfbeis, O. S., Optical Sensors for Determination of Heavy Metal Ion. Mikrochim Acta 126, 177-192, 1997: Walkup, G. K. & Imperiali , B., Design and Evaluation of a Peptidyl Fluorescent Chemosensor for Divalent Zinc, J. Am. Chem. Soc. 118, 3053-3054, 1996; Deo, S. & Godwin, H. A., A Selective Ratiometric Fluorescent Sensor for Pb²⁺, J. Am. Chem. Soc. 122, 174-175, 2000. These ion sensors are usually composed of an ion-binding motif and a fluorophore. Metal detection using these fluorescent chemo-sensors relies on the modulation of the fluorescent properties of the fluorophore by the metal-binding event. Detection limits on the level of micromolar and even nanomolar concentrations have been achieved for heavy metal ions including Zn²⁺, Cu²⁺, Hg²⁺, Cd²⁺ and Ag⁺.

Previous lack of understanding of the photo-physics of nanoparticles conjugated with antibodies bound to quenchers and exposed in order to target biological contaminants has limited the use of nanoparticles for real time detection and identification of specific biological contaminants. The barriers to solving the problem were: limited knowledge of spectral interactions among nanoparticles, biological contaminants and quenchers; steady state fluorescence properties of nanoparticles' antibody conjugates were not quantified; and dynamic changes in fluorescence properties were unknown.

Research completed to obtain fundamental knowledge on the fluorescence spectrum of nanoparticles and quenching phenomena analyzed steady state fluorescence, lifetime fluorescence and the effect of environmental variables such as concentration, pH, temperature and time. Specific research was accomplished by: analysis of distance maintained between nanoparticles and quenchers and the spectral criteria necessary for energy transfer and quenching in single quenching and dual quenching schemes; understanding of quenching and energy transfer phenomena in quenched nanoparticle biological contaminants using biological representative surrogates/antibody systems; and understanding of quenching mechanisms necessary to achieve consistent spectral signatures for detection and identification of a number of biological contaminants of interest.

An inexpensive system with acceptable false alarm rates is needed to decrease detection time, preferably to less than three minutes, to identify biological contaminants, in particular airborne types. Actively controlled electro-mechanical systems for detecting biological contaminants, such as those incorporated in heating, ventilation and air conditioning (HVAC) controls, may not be operable in an emergency situation. Having a “self-contained” (autonomous) capacity to rapidly detect and identify biological contaminants protects both personnel and assets.

### BRIEF DESCRIPTION OF DRAWINGS

FIG. 1 represents the basic interactions at the molecular level that result in the ability to detect and identify biological contaminants in fluids.

FIG. 2 is a graph showing the effects of varying concentrations of BHQ-2 quencher incubated with 4 μM of Qdots® for 30 minutes in the dark as measured on a fluorometer.

FIG. 3 graphs experimental versus predicted values of Förster's Distance for quenching assays.

FIG. 4 is a graph of atomic units (AU) versus wavelength in nanometers (nm) that summarizes the response of E. coli 0157H7 heat killed cells sensed by 605 nm Qdots®, demonstrating that concentrations of 10² colony forming units (cfu's) may be consistently and accurately measured with an incubation time of less than three minutes.

FIG. 5 is a graph of AU versus cfu's/mL illustrating that a FRET-based protocol not only alerts to the presence of a target, but also quantifies the amount of cfu's present.

FIG. 6 is a block diagram of a system, and variants thereof, which may be employed as select embodiments of the present invention incorporating fluoro-biosensors of select embodiments of the present invention.

FIG. 7 is a simplified depiction of a concentrator, “inoculated” substrates, light source and “stimulated” emissions as may be present in select embodiments of the present invention.

FIG. 8 depicts the response time of a representative embodiment of the present invention to the presence of two different concentrations of a targeted bio-contaminant.

### DETAILED DESCRIPTION

Select embodiments of the present invention comprise a sensor system that detects and identities biological contaminants (bio-contaminants) of interest, in particular airborne pathogens, preferably after an incubation time of less than about three minutes with the pathogens. Select embodiments of the present invention are based on a FRET reaction between fluorescing nanoparticles and a quencher that inhibits fluorescence in the absence of the one or more bacteria types of interest (bio-contaminant targets) in the sampled environment. That is, if there is no “active” bio-contaminant target in the “vicinity” (evanescent field) of the sensor, then fluorescence of the analog of the target is suppressed.

Select embodiments of the present invention also comprise a method of implementing the detection of bio-contaminant targets both individually and as a “multiplexed” system, the latter capable of detecting multiple bio-contaminant targets in a single solution.

In select embodiments of the present invention, nano-scale fluoro-biosensors detect and identity bio-contaminant targets, in particular targets that are airborne. The biosensor comprises: fluorescing nanoparticles; antibodies of one or more specific targets conjugated to the fluorescing nanoparticles: analogs of one or more targets; organic quencher molecules mixed with the analogs and conjugated fluorescing nanoparticles, such that the antibodies are covalently bonded to the fluorescing nanoparticles and the organic quencher molecules are attached to the analogs, and such that the fluorescing nanoparticles, antibodies, analogs, and organic quencher molecules are established on a substrate; one or more illuminators, such that an illuminator provides light at a pre-specified wavelength, λᵢ, to excite the fluorescing nanoparticles. those fluorescing nanoparticles attached to a first target providing one or more fluorescent responses at a second wavelength, λₙ, wherein i and n are integers, and such that the organic quencher molecules suppress fluorescing of the fluorescing nanoparticles associated with the analogs; a housing containing the conjugated fluorescing nanoparticles, the analogs and the organic quencher molecules, such that the housing permits illumination of the contents of the housing by the illuminator, and such that the housing permits entry of airborne targets.

In select embodiments of the present invention, a system detects, identifies and provides warning of bio-contaminant targets, in particular targets that are airborne. The system incorporates a nano-scale fluoro-biosensor comprising:fluorescing nanoparticles; antibodies of the targets conjugated to the fluorescing nanoparticles; analogs of the targets; organic quencher molecules mixed with the analogs and conjugated fluorescing nanoparticles, such that the antibodies are covalently bonded to the fluorescing nanoparticles and the organic quencher molecules are attached to the analogs, and such that the fluoro-biosensor is established on a substrate. The system further comprises: one or more illuminators, such that each illuminator provides light at a pre-specified wavelength, λᵢ, to excite at least the fluorescing nanoparticles, those fluorescing nanoparticles attached to a first target providing a fluorescent response at a second wavelength, λₙ, wherein both i and n are integers, and such that the organic quencher molecules attached to the analogs suppress fluorescing of the fluorescing nanoparticles associated with the analogs; a housing containing the conjugated fluorescing nanoparticles, analogs and organic quencher molecules, such that the housing permits illumination of its contents by the illuminator, and such that the housing permits entry of one or more targets; a controller; a receiver/processor in operable communication with the controller and the fluoro-biosensor, such that the receiver/processor receives the fluorescent response at a wavelength, λₙ, and such that the receiver/processor translates the received response into a signal for further processing; a clock for providing timing to at least the controller and illuminator; a means for warning a user, the means for warning in operable communication with the controller; a database and recorder in operable communication with the controller; wherein the system maintains a false alarm rate below a pre-specified maximum.

In select embodiments of the present invention, the nano-scale fluoro-biosensor system further comprises a receiver for receiving the fluorescent response at a wavelength, λₙ. The receiver translates the received response into a signal capable of being further processed. In select embodiments of the present invention, this signal ranges from one specific wavelength λₛ, to multiple output signals of unique wavelengths, λₙ, to enable the detection of multiple bio-contaminant targets in a single solution. In select embodiments of the present invention, as many as eight output signals of differing wavelength, λₙ, may be processed. In select embodiments of the present invention, the receiver/processor comprises one or more fluorometers.

In select embodiments of the present invention, the system comprises one or more warning indicators for alerting a user to the presence, of one or more targets in less than about three minutes.

In select embodiments of the present invention the system comprises one or more power supplies. In select, embodiments of the present invention, the one or more power supplies comprise one or more batteries.

In select embodiments of the present invention, the system comprises means for responding to a warning, such as an automatic shut off switch for air handling equipment, from the means for warning, such as a flashing light or siren.

In select embodiments of the present invention, the means for responding comprises one or more communication paths to disable an air handling system.

In select embodiments of the present invention part of the system is incorporated in an integrated circuit.

In select embodiments of the present invention the system incorporates means for an operator of the system to operate the system without the controller.

In select embodiments of the present invention the system comprises means for the system to operate without the controller.

In select embodiments of the present invention, the method provides means for responding to a warning, such as an automatic shutoff, from the means for warning, such as a bell or strobe.

In select embodiments of the present invention, the means for responding comprises a communications path to disable an air handling system.

In select embodiments of the present invention, the method provides means for an operator of the system to operate the system without the controller, such as manual override.

In select embodiments of the present invention, the method provides means for the system to operate without the controller, such as a backup system that may be remotely located.

In select embodiments of the present invention the method provides for incorporating part of the system in an integrated circuit.

In select embodiments of the present invention, the method further comprises providing multiple types of analogs, antibodies, and organic quenchers, and fluorescent nano-particles of differing emission wavelengths, to allow warning of the presence of multiple types of targets while maintaining a false alarm rate below a pre-specified maximum.

Select embodiments of the present invention allow “multiplexed detection” by employing multiple types of fluorescing nano-particles in an array of substrates. Each of the individual substrates in the array comprises a different type of fluorescing nano-particles in “solution,” preferably aqueous. Each substrate in the array may be employed to detect and identify a specific bio-contaminant from a possible group of like bio-contaminants. In select embodiments of the present invention, equal portions of air from a concentrator are supplied to each substrate through dedicated tubing that allows “bubbling” of the concentrated sample through the solution on each substrate.

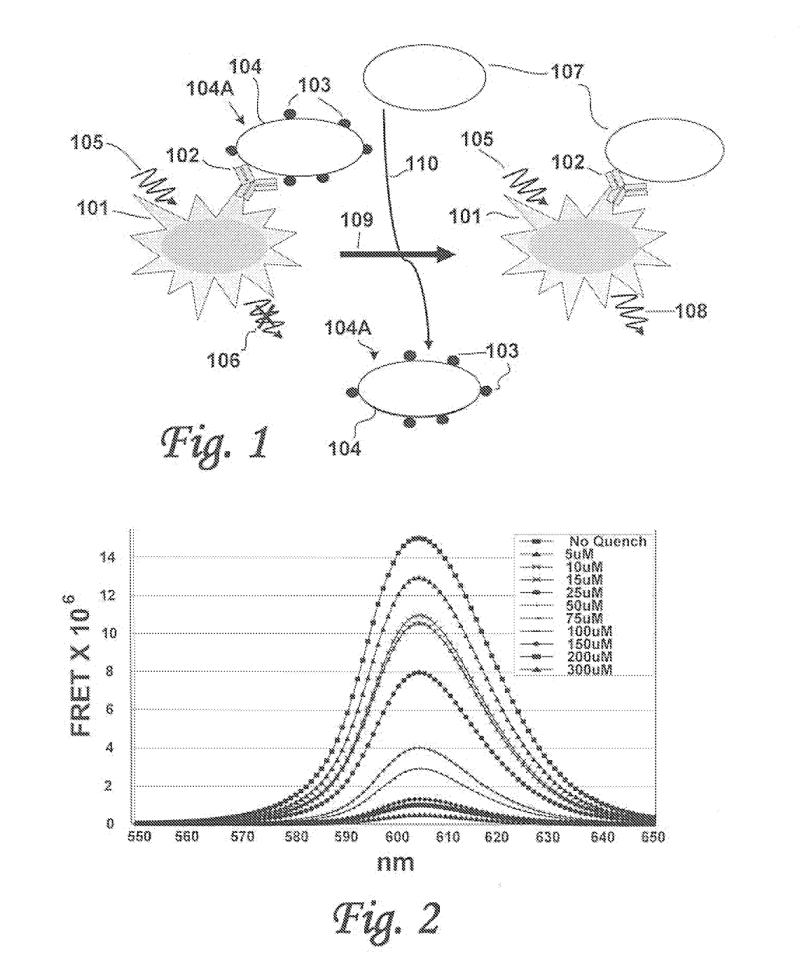

Refer to FIG. 1, in which fluorescent nanoparticles 101 (some are termed “Quantum Dots” in the literature and Qdots® by a particular vendor) are conjugated to targeted antibody fragments 102 specific to a bio-contaminant target such as E. coli. A quenching dye 103 is used to label an analog 104 (such as heat killed E. coli) of a bio-contaminant target 107 (such as E. coli). The specific antibody fragment 102 is covalently bonded to a fluorescent nanoparticle 101 which may comprise multiple nanoparticles responding at different secondary wavelengths, λₙ. In select embodiments of the present invention, the nanoparticles 101 are continuously excited by visible radiation 105. i.e., at wavelengths between about 400 and about 700 nanometers (nm), in the general case at about 400 nm. Coupling of the now nanoparticle-labeled antibody fragment 102 and a quencher (dye)-labeled bio-contaminant analog 104A. (usually within 10 nm) facilitates production of appreciable FRET based quenching 106. The quenching 106 significantly decreases fluorescence (emission) intensity. A bio-contaminant target 107 is detected 109 when it displaces 110 the quencher-labeled bio-contaminant analog 104 for which FRET has been suppressed. That is, the fluorescence emissions are restored only for the actual bio-contaminant target 107, not its analog 104. The emitted fluorescence 108 is dependent on the concentration of actual bio-contaminant targets 107, i.e., an increase in fluorescence from the nanoparticles 101 at a characteristic wavelength, e.g., 605 nm for contaminants such as E. coli 0157H7. The emitted fluorescence 108 is detected by an opto-electronic detector (not shown separately in FIG. 1) tuned to receive the pre-specified wavelength of fluorescent emission from the bio-contaminant target 107. This detection may be used to trigger an alarm to warn of the existence of the bio-contaminant target 107. For example, select embodiments of the present invention may be integrated into a warning system incorporated in a heating, ventilation, and air conditioning (HVAC) system.

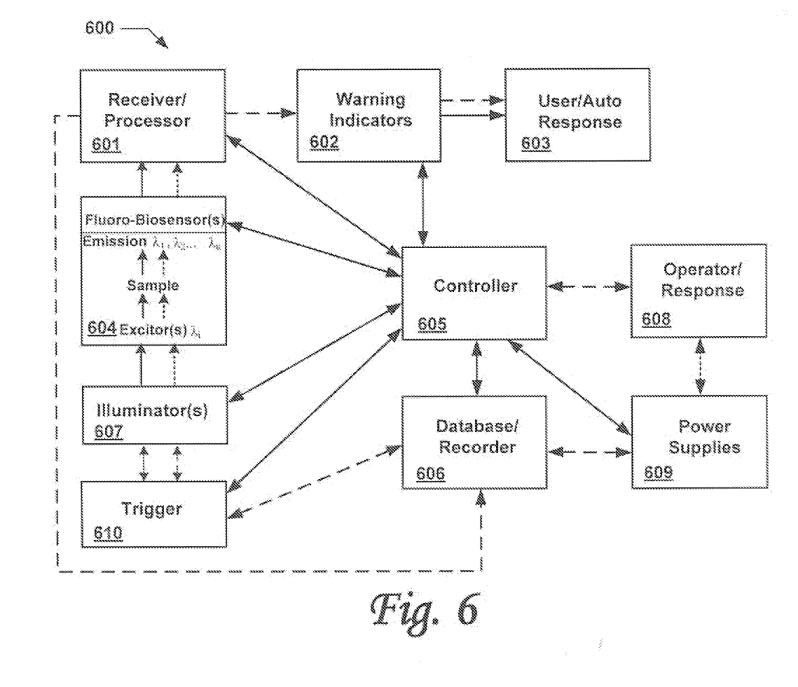

In its normal (dormant) state the immobilized analog 104A will be bound to the “tagged” antibody 102 and the proximity of a quencher (dye) 103 and fluorescent nanoparticles (e.g., Quantum Dots) 101 inhibits 106 fluorescence via FRET. As the bio-contaminant target 107 is approached 109 by the fluorescing nano-particles, “normal” fluorescence 108 is enabled, and a sufficient response initiated for receipt by the pre-specified threshold of a receiver (not shown separately in FIG. 1), such as a fluorometer. The receiver 601 (FIG. 6) may be in operable communication with a processor or may be a receiver/processor 601 suitable for further converting the received fluorescent response to a warning to be initiated by-warning indicators 602 (FIG. 6). As indicated by the arrow 110, without the fluorescent nanoparticles 101, the bio-contaminant target 107 draws no reaction from the quenched analog 104A.

The proximity (distance) required for “reasonable” quenching is termed the “Förster Distance” and is representative of the span at which 50% of emissions from the nanoparticles 101 will be absorbed by a neighboring quencher-labeled bio-contaminant target analog 104A. When a test sample containing unlabeled E. coli is added to the conjugate, equilibrium reactions cause the displacement on the antibody 102 of the quencher-labeled analog target 104 for the unlabeled bio-contaminant target 107 from the test sample. Those nanoparticles 101, no longer adjacent to a quencher-labeled bio-contaminant target analog 104A, freely fluoresce 108. Quantitative data may thus be collected on the concentration of the bio-contaminant target 107.

Fluoro-sensors of embodiments of the present invention are based on FRET and have a low false alarm rate that is dependent on the specificity (polyclonal vs. monoclonal) of the fragmented antibody 102. Fluoro-sensors of select embodiments of the present invention have the potential to selectively detect different species of bacteria from the same family. In select embodiments of the present invention. Qdots® 101 and bacteria-specific antibodies 102 detect the E. coli in aqueous solutions. For example, the pathogenic E. coli strain may be detected as well as commonly encountered nonpathogenie E. coli strains. Because of the innately strong selectivity of antibodies 102, judicious selection of antibodies 102 for use in select embodiments of the present results in an acceptable number of false positives, i.e., a low false alarm rate.

By incorporating fluorescent nano-particles of different emission wavelengths, select embodiments of the present invention are capable of detecting any of a number of targeted biological contaminants from a broad spectrum of possible contaminants instantaneously based on the wavelength of the emitted fluorescence.

### EXAMPLE

Tests of select embodiments of the present invention indicate detection as low as 1×10² cfu's/mL for E. coli samples. In select embodiments of the present invention, a system employing FRET reactions between fluorescent nanoparticles 101 and organic quencher molecules 104A detects and identifies E. coli 0571H7, often used as a model biological contaminant. Further, pathogens were detected and identified in less than three minutes. Select embodiments of the present invention detect and identify a broad range of viral and bio-contaminants with a degree of specificity and selectivity on the order of 10² cfu's/mL as previously mentioned. Furthermore, by incorporating fluorescent, nanoparticles 101 of different emission wavelengths, λᵢ, select embodiments of the present invention may implement a multiplexing immunoassay.

E. coli antibody fragments 102 conjugated to fluorescing nanoparticles 101 (Qdots®, Quantum Dot Corporation, 4030 Sabian Way, Palo Alto Calif. 94303) with an emission at 605 nm in concert with a quencher 103 (Black Hole Quencher (BHQ-2) from BHQ-2 Quencher, Bio-Synthesis, Inc., 612 East Main Street, Lewisville, Tex. 75057) were used. In select embodiments of the present invention, any one of multiple wavelengths, λᵢ, may be used to illuminate in a multiplexed embodiment. Qdots® 101 are fluorophores capable of emitting high fluorescent energy, thus capable of interacting with multiple acceptors. Fluorescing nanoparticles 101 and quenchers 103 are chosen so that emission and absorbance ranges overlap as closely as possible to insure maximum FRET suppression. While concentrations were able to be established for labeling with the Qdots® 101, there was no set concentration of quencher 103 established so an ideal concentration had to be determined. A BHQ-2 quencher 103 was used to determine the effects of varying the concentration of quencher 103 to determine the concentration needed to effectively quench 605 nm Qdots® 101.

FIG. 2 shows test results from varying concentrations of BHQ-2 quencher 103, incubated with 4 μM of Qdots® 605 nm for 30 minutes in the dark. This time is in excess of the time needed for the reaction to progress but was extended to 30 minutes to allow the quencher 103 to completely react. The BHQ-2 quencher 103 was chosen for its strong absorbance around 605 nm, the fluorescence wavelength of the Qdots® 101. In the above scenario the acceptors are the quenchers 103. The rate at which these quenchers 103 absorb the fluorescence 108 is dependent on the concentration of both the Qdots® 101 and the quenchers 103. A fluorometer was used to measure response to determine what concentration of quencher 103 is optimum, i.e., that adequately diminishes the fluorescence 108 while maintaining a background baseline of fluorescence 108. As seen in FIG. 2, the ability of the BHQ-2 quencher 103 to quench is dependent on the concentration of quencher 103. To maximize FRET-based quenching while avoiding quenching of a positive signal, 25 μM concentrations were selected. This concentration was also used to calculate overall FRET efficiency.

The decrease in signal is due to transfer of energy from a donor, e.g., a 605 nm Qdot® 101, to an acceptor, e.g., a BHQ-2 quencher 103, in lieu of being emitted as a photon of light seen as FRET. This can be expressed in the following equation:

$$E = \frac{{nR}_{0}^{6}}{{nR}_{0}^{6} + {r}^{6}} \qquad ( 1 )$$

where:

E=rate of FRET

n=number of acceptors interacting with a donor

R₀=Förster distance

r=centro-symmetric equal separation distance between donors

Eqn. (1) states that the rate of FRET, E, is dependent on the number of acceptors, n, interacting with a donor molecule and the Förster distance, R₀, and is proportional to the number of acceptors, n, times the Förster distance, R₀, to the 6^{th} power plus the distance of centro-symmetric equal separation between donor molecules, r to the 6^{th} power. In order to find this answer the Förster distance, R₀, must first be determined. The Förster distance, R₀, is defined as the optimal distance at which FRET efficiency is estimated to be at about 50%, as given by:

$${R}_{0} = {( {BQ}_{D} I )}^{\frac{1}{6}} = {[ \frac{9000 ( ln 10 ) {k}_{p}^{2} {Q}_{D} I}{{N}_{A} 128 {π}^{3} {n}_{D}^{2}} ]}^{\frac{1}{6}} \qquad ( 2 )$$

where:
B=a function of Avogadro's number;
Q_{D}=quantum yield of nanoparticles (Quantum dots);
I=the integral of spectral overlap between acceptor absorption and donor emission;
N_{A}=refractive index of the medium;
n_{D}=number of acceptors interacting with a donor
κₚ=parameter that depends on the dipole orientation of the molecules

Information on dipole orientation, as obtained from previous experimentation and publications on randomly oriented dipoles, suggests κₚ be set to 0.66. The value used for n_{D} was set at 36 to provide an estimate of the separation distance from the center of the fluorescent nanoparticles 101 to the acceptor quencher 103. The distance is approximately 77 Å from center to surface for 605 nm Qdots®.

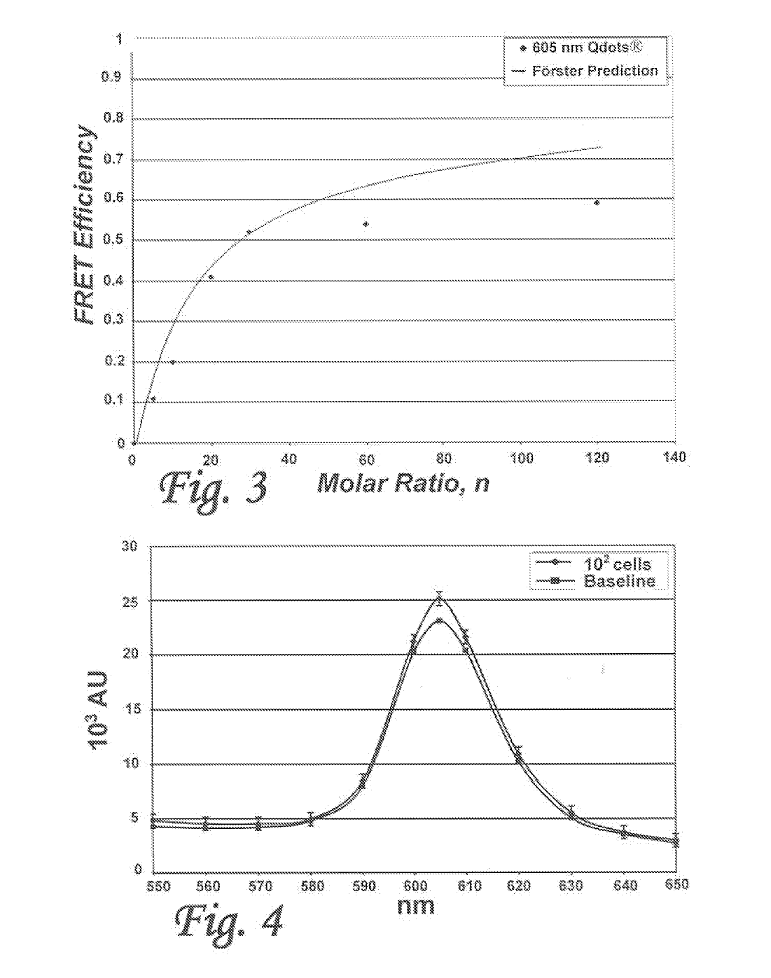

These values along with constants and the experimentally available values (molar concentrations, refractive index of the medium, etc.), facilitated construction of FIG. 3 comparing FRET efficiency vs. molar ratio, n, for the Förster prediction with empirical results using the Qdots® at 605 nm. For ratios of less than 40 (FRET efficiency less than or equal to about 50%), the two methods are comparable. This shows the close fit of empirical data with predicted values from the Förster distance equation. At higher concentrations of quenchers 103. i.e., ratios above about 40, empirical values digress from the “idea” FRET efficiency, however, the latter assumes no loss of energy in the system. The discrepancy is probably due to an increased molar ratio, n, of quenchers 103 to nanoparticles 101. There is a finite surface area available on each nanoparticle 101 and once that area is occupied, maximum quenching occurs. Thus, no matter how much BHQ-2 quencher 103 is added at this point, the amount of quenching does not increase.

IgG monoclonal antibodies 102 were selected for use over other possible ligands because of their exceptional properties. Antibodies 102 may be developed to meet most sensing needs. Furthermore, when specified adequately, an antibody 102 is very sensitive to its target, thus a properly designed system practically eliminates false positives. An antibody 102 may avoid false positives even in the case of two very closely related bacterial strains, e.g., E. coli 0571H7, a foodborne pathogen, was selected specifically over other E. coli strains in a series of tests.

In selected experiments, an IgG monoclonal mouse derived antibody 102 was used. These antibodies 102 were fragmented in an immobilized pepsin digest developed by Pierce Biotechnology, Inc., P.O. Box 117, Rockford, Ill. 61105. Pepsin is an enzymatic molecule that is particularly adept at cleaving peptides at the aromatic amino acids, phenylalanine, tryptophan, and tyrosine. This protocol generates the F(ab₂) fragments of IgG antibodies 102 employed in select testing and numerous small Fc antibody fragments 102 that are then separated by a 50,000 molecular weight cut off (MWCO) filter. The resulting solution containing the F(ab₂) antibody fragments 102 was concentrated to approximately 1.5 mg/mL for use in the conjugation step.

Luminescent colloidal semiconductor nanocrystals 101 were chosen over other commercially available fluorescent nanoparticles such as fluorescent dyes or fluorophores because of their exceptional intensity, resistance to bleaching, and large Stokes shifts. These characteristics all support a highly sensitive system that facilitates detection of bio-contaminant targets 107 present in low concentrations. The nanocrystals 101 are nanoparticles with a Zn-coated CdSe core and are available from Molecular Probes Division, Invitrogen Corporation, 1600 Faraday Avenue, Carlsbad, Calif. 92008. The size of the core of a nanocrystal 101 is related to the emission wavelength of the nanocrystal 101. Nanocyrstals 101 function similarly to conventionally employed fluorescent dyes in converting absorbed light of one wavelength, λᵢ, into emitted light of another wavelength, λₙ, however, the yield from nanocrystals 101 is greater and more chemically stable. Since nanocrystals 101 are available in a wide variety of colors, i.e., wavelengths, separate conjugations may be developed in a single multiplex Setup. This multiplexing permits several bio-contaminant targets to be sensed simultaneously, each represented by a different response wavelength, λₙ.

Using the hetero-bifunctional crosslinker, 4-(maleimidomethyl)-1-cyclohexanecarboxylic acid N-hydroxysuccinimide ester (SMCC), 605 nm Qdots® 101 were activated to yield a maleimide-nanocrystal surface. Concurrently, dithiothreitol (DTT) was used to reduce the antibody 102, thereby exposing free sulfhydryls. The chemical treatment of the antibody 102 and conjugate allowed the two to covalently bond. The protocol and materials used are from the Quantum Dot Corp.'s Antibody Labeling Kit. This protocol is available in a wide assortment of Qdots® 101. When paired with different antibody fragments 102 these protocols may be used to detect and identify multiple bio-contaminant targets 107 in the same reaction.

Quenchers 103 function by absorbing light within a range of wavelengths and converting it into heat, thus dampening fluorescence 108. Conventional FRET detection is done either through quenching of a signal (not molecules) or by conversion of a signal to an alternate wavelength via pairing. In quenching nanoparticles 101, the actual fluorescence 108 is quenched by a neighboring particle. To pair molecules rather than signals, the fluorescence of one fluorescent nanoparticle 101 is absorbed by a neighboring fluorescent nanoparticle 101 and re-emitted at a different wavelength. Thus, in select embodiments of the present invention, detection in a quenching protocol depends on an increase in net fluorescence 108 at a pre-specified wavelength, λₙ, whereas detection in a pairing protocol depends on a color, i.e., wavelength, shift. Note that a simple pairing protocol is not suitable for a multiplexing system because of cross-talk between fluorophores that increases as additional fluorophores of different wavelengths are added.

Solid E. coli were obtained from KPL Inc., 910 Clopper Road, Gaithersburg, Md. 20878, in a concentration of 1 mg containing about 3.0×10⁸ cfu's. This was re-suspended in 2 ml of 1× PBS. Quencher BHQ-2 103 was then added to 1 mL of this E. coli solution at a final concentration of 25 μg/mL as suggested by the manufacturer and incubated for two hours in darkness. The remaining E. coli solution was used for dilutions of the bio-contaminant target 107, ranging from 10₈ to 10₂ cfu's for measurement with the Qdot®—F(ab₂) fragment conjugate system. After the two-hour incubation of the E. coli with BHQ-2 quencher 103, the quenched E. coli 104A were washed of unbound quencher 103 by two washes with 1 mL of 1× PBS and centrifugation for two minutes at 13,000 rpm. This mixture was re-suspended in 75 μL of 1× PBS and added to 75 μL of Qdot®—F(ab₂) conjugate, and reacted tor 30 minutes in the dark. After 30 minutes this solution was washed with 1× PBS as before and re-suspended in 500 μL of 1× PBS. Each serial bacterial dilution (10⁸ down to 10²) of heat killed E. coli 104 contained a volume of 450 μl to which 50 μl of the quenched Qdot®—F(ab₂) solution was added. The reaction ran for less than 3 minutes and the results were read on a Fluoro-Max fluorometer with Xenon lamp excitation at 400 nm. Each solution was excited at 400 nm and any fluorescence 108 from 500 to 700 nm was measured, with a focus at 605 nm.

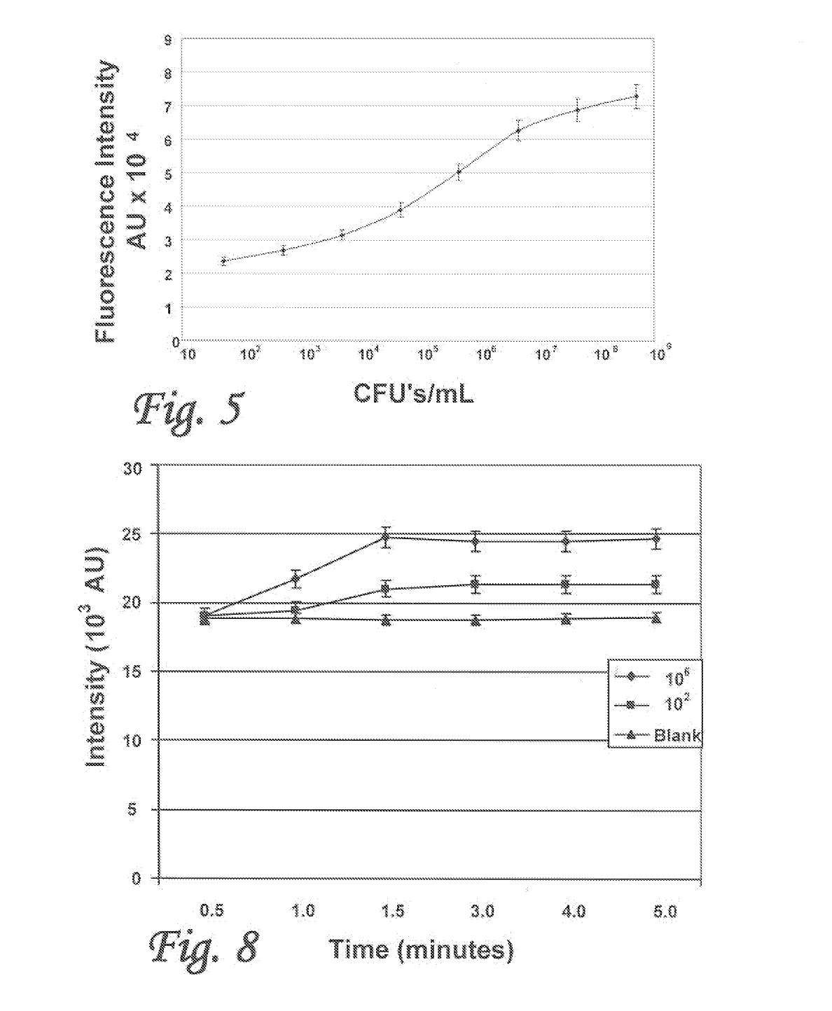

Refer to FIG. 5. Select embodiments of the present invention are capable of a detection threshold of 10² cfu's/mL, well within diagnostic needs for the detection of bio-contaminant targets 107 such as food-borne pathogens. Further, as can be seen from the graph of FIG. 5, the intensity of fluorescence 108 is dependent on the concentration of bio-contaminant target cells 107 present, allowing for a quantitative calculation of the amount of bio-contaminant target 107 present. The ability to detect not only the bio-contaminant target 107 present but also the concentration allows this highly adaptable protocol to be applied to a number of bio-contaminant targets 107. Through the use of multiple nanoparticles 101 of different emission wavelengths, λᵢ, a single device may be provided that simultaneously scans for a “catalog” of bio-contaminant targets 107.

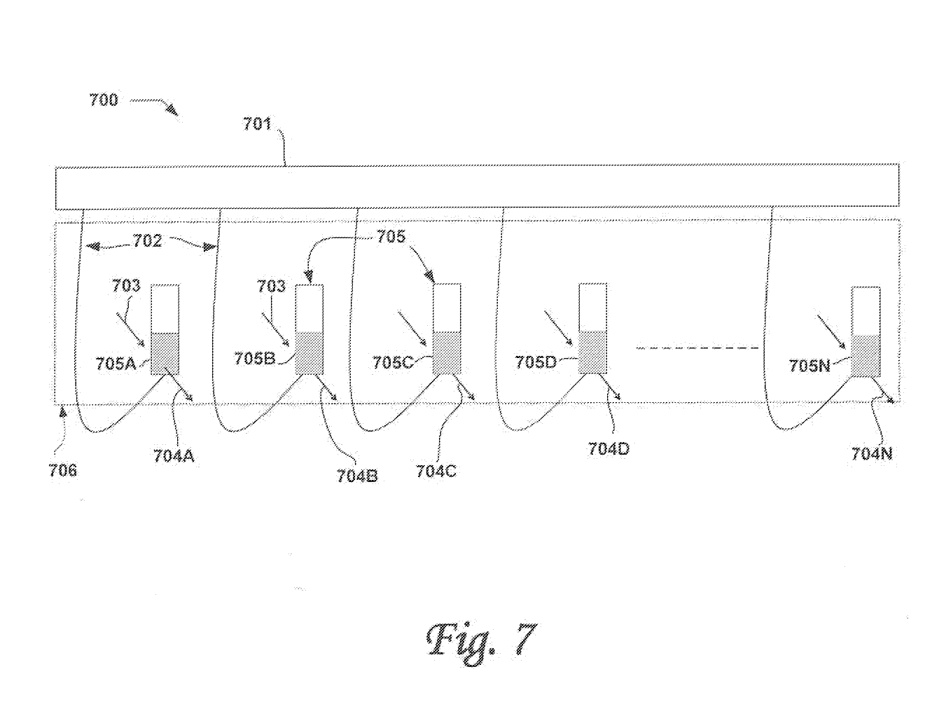

Select embodiments of the present invention comprise an autonomous closed loop system 600 for detecting biological contaminants. Select embodiments of the present invention comprise a housing 701 (FIG. 7) incorporating fluoro-biosensors 604 on a substrate 704 (FIG. 7) for fluorophores 101, antibodies 102, quenchers 103 and analogs 104, the substrate open to the environment desired to be sampled, a controller 605, a recorder 606 with optional database, a receiver 601 with optional processor, one or more illuminators 607, a clock 610 for timing, a power source 609 including an optional backup, one or more warning indicators 602, and a mechanism providing a backup mode.

Refer to FIG. 6, representing a system 600 (and variants thereof) that may be employed as select embodiments of the present invention incorporating fluoro-biosensors 604 of select embodiments of the present invention. Note that more than one “exciter” at initial wavelength, λᵢ, may be employed to “excite” a sample from an air concentrator, and multiple emissions. λ₁, λ₂, . . . λₙ may be induced depending on both the number of initial exciter wavelengths, λᵢ, and the number of types of targeted bio-contaminants 107. In select embodiments of die present invention, the controller 605 functions to operate the system 600 autonomously, communicating bi-directionally (as indicated by the solid arrows of FIG. 6) with power supplies 609 that may include back-up sources (not shown separately), a database/recorder 606 for both providing data on archived activity and recording present activity, a trigger 610 for synchronizing events and establishing time of activity, an illuminator 607 to “excite” the fluoro-biosensors 604, a receiver/processor 601 to both receive and process responses from the fluoro-biosensors 604, and warning indicators 602 that may be visual, aural or both, for alerting a user/automated response system 603, such as a resident of a building protected by the system 600 or an automated system to counteract the bio-contaminant target 107, or both.

An alternate or “backup” mode may provide for an operator/response system alternate 608 that interacts with the controller 605. in particular, when the controller 605 may be experiencing technical problems, as indicated by the dashed arrows of FIG. 6. The operator/response system alternate 608 may be remotely located or an alternate “response” may be initiated by a remote backup controller in case of failure or degradation of the primary controller 605. In that case the power supplies 609, including any battery backup would be directed to activate an alternate path indicated by the dashed lines of FIG. 6. Thus, in the alternate or backup mode, the database/recorder 606 serves as a link to the trigger 610 as well as a feedback link from the receiver processor 601 through the link to the power supplies 609 to the operator/response system 608. The trigger 610 would still interface with the illuminators(s) 607 and the illuminator(s) 607 would, excite the fluoro-biosensor(s) 604, the response to which would be received and processed by the receiver/processor 601 that would then provide the input directly to the warning indicators 602 to the user/automated response 603, all without the use of the primary controller 605.

Refer to FIG. 7, a simplified representation of an embodiment 700 of the present invention, an array 706 of detectors 705 fed via tubes 702 from a particle concentrator 701. Each of the “fed” substrates 705 incorporates ihc fluorescing nano-particles 101 in a solution 705A, B, C, D, N on the substrates 705 and quenched analogs 104A. For example, solution 705A may contain fluorescing nano-particles 101 with only spore-forming bacteria, antibodies attached, solution 705B may contain fluorescing nano-particles 101 with only vegetative bacteria antibodies attached, solution 705C may contain fluorescing nano-particles 101 with only toxin antibodies attached, and solution 705D may contain fluorescing nano-particles 101 with only viral antibodies attached. Also depicted in FIG. 7 is the illumination 703 from the light source(s) 105 and resultant induced emissions 704A, B, C, D, N that may be collected by an optical receiver, such as a fluorometer, for further processing as described above. As shown, a source of airborne particles enters the tubes 702 from the concentrator 701 and is bubbled through the solution(s) 705A, B, C, D, N on the substrate(s) 705. The airborne particles may be provided as a sample from a particle concentrator 701 located in the airstream of an air handling system such as may be associated with an HVAC system (not shown separately).

Refer to FIG. 8 depicting the response time of an embodiment of the present invention for detecting a targeted contaminant at each of two (2) concentrations, 10² and 10⁶ cfu's, as well as the response of a “blank” sample. From this data, it is evident that even at low concentrations, select embodiments of the present invention are able to detect and identify a targeted bio-contaminant in about 1.5 minutes and in about one minute at the higher concentrations that may be expected under real world conditions, while correctly ignoring the blank sample to achieve a low rate of false alarm.

The abstract of the disclosure is provided to comply with the rules requiring an abstract that will allow a searcher to quickly ascertain the subject matter of the technical disclosure of any patent issued from this disclosure. (37 CFR §1.72(b)). Any advantages and benefits described may not apply to all embodiments of the invention.

While the invention has been described in terms of some of its embodiments, those skilled in the art will recognize that the invention can be practiced with modifications within the spirit and scope of the appended claims. For example, although the system is described in specific examples for detecting and identifying airborne bio-contaminants in buildings, it may be used for any type of fluid contaminants and thus may be useful in such diverse applications as manufacturing, food processing, agriculture, dairy farming, refining, re-cycling, remediating, food preparation, and the like. Bio-contaminants of interest may be from manufacturing plants or food handling facilities, schools, hospitals, universities, multi-family residences, public conveyances, and the like. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. Thus, it is intended that all matter contained in the foregoing description or shown in the accompanying drawings shall be interpreted as illustrative rather than limiting, and the invention should be defined only in accordance with the following claims and their equivalents.

We claim:

1. A nano-scale fluoro-biosensor that detects and identifies bio-contaminant targets, in particular said targets that are airborne, comprising:
fluorescing nanoparticles;
antibodies of said targets conjugated to said fluorescing nanoparticles;
analogs of said targets;
organic quencher molecules mixed with said analogs and said conjugated fluorescing nanoparticles,
wherein said antibodies are covalently bonded to said fluorescing nanoparticles and said organic quencher molecules arc attached to said analogs, and
wherein said fluorescing nanoparticles, said antibodies, said analogs, and said organic quencher are established on a substrate;
at least one illuminator,
wherein said illuminator provides light at a pre-spccified wavelength. λᵢ, to excite at least said fluorescing nanoparticles, those said fluorescing nanoparticles attached to a first said target providing at least one fluorescent response at a second wavelength, λₙ, wherein both i and n are integers, and
wherein said organic quencher molecules attached to said analogs suppress fluorescing of said fluorescing nanoparticles associated with said analogs;
a housing containing at least said conjugated fluorescing nanoparticles, said analogs
and said organic quencher molecules,
wherein said housing permits illumination of the contents of said housing by said illuminator, and
wherein said housing permits entry of at least airborne said targets.

2. A system that detects, identifies and provides warning of bio-contaminant targets, in particular said targets that are airborne, comprising:
a nano-scale fluoro-biosensor comprising:
fluorescing nanoparticles;
antibodies of said targets conjugated to said fluorescing nanoparticles;
analogs of said targets;
organic quencher molecules mixed with said analogs and said conjugated fluorescing nanoparticles,
wherein said antibodies are covalently bonded to said fluorescing nanoparticles and said organic quencher molecules are attached to said analogs, and
wherein said fluoro-biosensor is established on a substrate;
at least one illuminator,
wherein said illuminator provides light at a pre-specified wavelength, λᵢ, to excite at least said fluorescing nanoparticles, those said fluorescing nanoparticles attached to a first said target providing a fluorescent response at a second wavelength, λₙ, wherein, both i and n are integers, and
wherein said organic quencher molecules attached to said analogs suppress fluorescing of said fluorescing nanoparticles associated with said analogs;
a housing containing at least said conjugated fluorescing nanoparticles, said analogs and said organic quencher molecules,
wherein said housing permits illumination of the contents of said housing by said illuminator, and
wherein said housing permits entry of at least said targets that are airborne;
a controller;
a receiver/processor in operable communication with at least said controller and said fluoro-biosensor,
wherein said receiver/processor receives at least said fluorescent response at a wavelength, λₙ, and
wherein said receiver/processor translates said received response into a signal for further processing;
a clock for providing timing to at least said controller and said illuminator;
a means for warning at least a user, said means for warning in operable communication with at least said controller;
a database and recorder in operable communication with at least said controller;
wherein said system maintains a false alarm rate below a pre-specified maximum.

3. The system of claim 2 said receiver/processor comprising at least one fluorometer.

4. The system of claim 2 further comprising at least one warning indicator for at least alerting a user to the presence of at least one said target in less than about three minutes.

5. The system of claim 2 further comprising at least one power supply.

6. The system of claim 5 said power supply comprising at least one battery.

7. The system of claim 2 further comprising means for responding to a warning from said means for warning.

8. The system of claim 7 said means for responding comprising at least a communications path to disable an air handling system.

9. The system of claim 2 further providing incorporating at least part of said system in an integrated circuit and incorporating said fluorescing nanoparticles, said antibodies of said targets, said analogs of said targets, and said organic quencher molecules in an aqueous solution.

10. The system of claim 2 further comprising means for an operator of said system to operate said system without said controller.

11. The system of claim 2 further comprising means for said system to operate without said controller.

12. A method for detecting, identifying and providing warning of bio-contaminant targets, in particular said targets that are airborne, comprising:
providing a system for detecting and identifying said targets, comprising:
a nano-scale fluoro-biosensor comprising:
fluorescing nanoparticles;
antibodies of said targets conjugated to said fluorescing nanoparticles;
analogs of said targets;
organic quencher molecules mixed with said analogs and said conjugated fluorescing nanoparticles,
wherein said antibodies arc covalently bonded to said fluorescing nanoparticles and said organic quencher molecules are attached to said analogs, and
wherein said fluoro-biosensor is established on a substrate:
providing at least one illuminator,
wherein said illuminator provides light at a pre-specified wavelength, λᵢ, to excite at least said fluorescing nanoparticles, those said fluorescing nanoparticles attached to a first said target providing a fluorescent response at a second wavelength, λₙ, different from said λᵢ, wherein n is an integer, and
wherein said organic quencher molecules attached to said analogs suppress fluorescing of said fluorescing nanoparticles associated with said analogs;
providing a housing containing at least said conjugated fluorescing nanoparticles,
said analogs and said organic quencher molecules,
wherein said housing permits illumination of the contents of said housing by said illuminator, and
wherein said housing permits entry of at least said targets that are airborne;
providing a controller;
providing a processor in operable communication with said controller;
providing a clock for providing timing to at least said controller and said illuminator;
providing a means for warning in operable communication with at least said controller and at least one user; and
providing a database and recorder in operable communication with at least said controller,
wherein said method maintains a false alarm rate below a pre-specified maximum.

13. The method of claim 12 further providing at least one power supply, said method further detecting and identifying said targets in less than about three minutes.

14. The method of claim 13 providing said power supply as at least one battery.

15. The method of claim 12 further providing means for responding to a warning from said means for warning.

16. The method of claim 15 said means for responding comprising at least a communications path to disable an air handling system.

17. The method of claim 12 further providing means for an operator of said system to operate said system without said controller.

18. The method of claim 12 further providing means for said system to operate without said controller.

19. The method of claim 12 further providing incorporating at least part of said system in an integrated circuit and incorporating said fluorescing nanoparticles, said antibodies of said targets, said analogs of said targets, and said organic quencher molecules in an aqueous solution.

20. The method of claim 12 further comprising an array for:
providing multiple types of said analogs, multiple types of said antibodies, and
multiple types of said organic quenchers, and
providing said fluorescent nano-particles of differing emission wavelengths,
wherein said method provides warning of multiple types of said targets while maintaining a false alarm rate below a pre-specified maximum.
